(19) United States
(12) Patent Application Publication
(10) Pub. No.: US 2026/0014222 A1
(43) Pub. Date: Jan. 15, 2026

(54) HERBAL COMPOSITION FOR A TREATMENT OF HYPERTENSION, HYPERLIPIDEMIA, DIABETES, AND CARDIOVASCULAR RISK FACTORS

(71) Applicant: Ancient Wisdom Medicine, LLC, NEWARK, DE (US)

(72) Inventor: Lurong P. Rosser, NEWARK, DE (US)

(21) Appl. No.: 19/259,007

(22) Filed: Jul. 3, 2025

Related U.S. Application Data
(60) Provisional application No. 63/669,251, filed on Jul. 10, 2024.

(51) Int. Cl. A61K 36/515; A61K 9/28; A61K 9/50; A61K 36/185; A61K 36/42; A61K 36/72; A61K 47/26

(52) U.S. Cl. CPC A61K 36/515; A61K 9/28; A61K 9/5042; A61K 36/185; A61K 36/42; A61K 36/72; A61K 47/26; A61K 2236/17; A61K 2236/37; A61K 2236/51

(57) ABSTRACT

The present invention relates to an herbal composition and method for a treatment and management of at least one of hypertension, hyperlipidemia, diabetes mellitus, and associated cardiovascular risk factors. The composition comprises standardized plant extracts derived from Rauvolfia serpentina, Hippophae rhamnoides seed, Citrullus lanatus seed, and Phyllanthus emblica fruit, in approximate weight proportions ranging from 20-40% for Rauvolfia serpentina, 20-40% for Hippophae rhamnoides, 10-30% for Citrullus lanatus, and 10-30% for Phyllanthus emblica. The invention also provides a method of preparing the composition involving harvesting, drying, grinding, extracting with selected solvents, concentrating, optionally fractionating, and formulating into oral dosage forms such as one of capsules, tablets, and powder sachets. The invention further includes methods for administering the composition to a subject in need thereof, optionally as an adjunct to conventional therapy.

### TECHNICAL FIELD

The present invention relates to an herbal composition and a method of preparation thereof for a treatment of chronic metabolic and cardiovascular conditions. More particularly, the present invention pertains to a synergistic formulation comprising plant extracts of Rauvolfia serpentina, Hippophae rhamnoides seed, Citrullus lanatus seed, and Phyllanthus emblica fruit, useful in prevention, and treatment of one or more health conditions including hypertension, hyperlipidemia, diabetes mellitus, and cardiovascular risk factors.

### BACKGROUND

Hypertension, commonly known as high blood pressure, is a chronic medical condition characterized by persistently elevated pressure in the arteries. It may develop gradually over time due to genetic predisposition, dietary habits, lifestyle factors, or as a result of secondary causes such as renal or thyroid disorders, and certain medications. If left untreated or poorly managed, hypertension can lead to severe cardiovascular complications including heart disease, myocardial infarction, and stroke. According to the World Health Organization (WHO), approximately 1.28 billion adults aged 30 to 79 globally suffer from hypertension, yet only about 42% of these individuals are diagnosed and treated, and merely 21% have their condition adequately controlled. Hypertension continues to be a leading contributor to premature mortality worldwide.

Hypercholesterolemia, or high cholesterol, is another prevalent metabolic disorder defined by elevated levels of total cholesterol and/or low-density lipoprotein (LDL) cholesterol in the bloodstream. It may arise from dietary patterns rich in saturated fats, genetic conditions such as familial hypercholesterolemia, physical inactivity, psychological stress, medication use, or comorbid health disorders. Persistent high cholesterol levels contribute to the development of atherosclerosis, a condition marked by the accumulation of lipid-rich plaques on arterial walls. Atherosclerosis significantly narrows the arteries and impairs blood flow, increasing the risk of coronary artery disease, heart attacks, and cerebrovascular events such as strokes.

Diabetes mellitus, particularly type 2 diabetes, is a widespread metabolic condition characterized by hyperglycemia resulting from insulin resistance or impaired insulin secretion. Diabetes itself is an independent and compounding risk factor for cardiovascular disease. It is known to exacerbate both hypertension and dyslipidemia, creating a synergistic pathological environment that accelerates vascular damage and cardiovascular complications. Together, hypertension, hyperlipidemia, and diabetes form a triad of interrelated conditions that markedly increase the overall burden of cardiovascular risk.

Conventional medical treatments for these conditions typically rely on synthetic pharmaceutical agents. Antihypertensive drugs include beta-blockers, angiotensin-converting enzyme (ACE) inhibitors, and calcium channel blockers. Cholesterol-lowering agents such as statins are widely prescribed to manage dyslipidemia. Antidiabetic drugs like metformin, sulfonylureas, insulin, and other hypoglycemics are routinely used for glycemic control. While effective, these synthetic medications are associated with several limitations and adverse effects. Long-term usage may lead to undesirable side effects such as fatigue, gastrointestinal disturbances, myopathy, hepatotoxicity, and, in some cases, serious systemic complications. Additionally, many patients require multiple drugs to manage coexisting conditions, which can result in polypharmacy, reduced patient adherence, drug-drug interactions, and increased economic burden. Certain drugs may exhibit diminishing efficacy over time, necessitating dose escalation or adjunctive therapy, further complicating treatment regimens. Moreover, many synthetic drugs primarily address symptomatic relief or laboratory parameters, without addressing the underlying systemic imbalance.

In view of these limitations, there is growing global interest in alternative or complementary treatment modalities, particularly those derived from natural sources. Herbal and plant-based interventions are increasingly sought for their holistic therapeutic potential, lower incidence of side effects, and auxiliary health benefits such as antioxidant, anti-inflammatory, and vasoprotective effects. Traditional medicinal systems have long utilized certain plants for managing cardiovascular and metabolic conditions.

For example, Rauvolfia serpentina (Indian snakeroot) has been extensively used in Ayurvedic medicine for its hypotensive effects, primarily attributed to the presence of reserpine and related indole alkaloids. Phyllanthus emblica (amla or Indian gooseberry) is recognized for its potent antioxidant profile and is known to support lipid regulation and glycemic control. Hippophae rhamnoides (sea buckthorn), particularly its seeds, contains flavonoids, tocopherols, and omega fatty acids that contribute to cholesterol modulation and cardiovascular protection. Citrullus lanatus (watermelon) seeds are a natural source of L-arginine and magnesium, nutrients that support nitric oxide-mediated vasodilation and improve glucose metabolism.

However, no existing single composition combines these specific natural ingredients in a manner that simultaneously targets hypertension, high cholesterol, diabetes, and related cardiovascular risks. There remains a need for an integrated, multi-herbal composition that can address these conditions together, providing a broad-spectrum therapeutic effect while avoiding the drawbacks of synthetic drugs. Accordingly, there is a need for a composition comprising a combination of natural ingredients to treat hypertension, high cholesterol, diabetes, and cardiovascular risk factors in a safe and effective manner.

### SUMMARY

This summary is provided to introduce the invention in a simplified form; it is not intended to delimit the scope of the invention, which is defined by the claims.

In one aspect, the present invention provides a therapeutic composition comprising a combination of natural ingredients for the treatment of one more health condition comprising health hypertension, high cholesterol, diabetes, and associated cardiovascular risk factors. The composition comprises extracts or bioactive fractions derived from four plant sources: a first extract derived from Rauvolfia serpentina (such as extracts from its roots, commonly known as Sarpagandha), a second extract derived from Hippophae rhamnoides seed (extracts from sea buckthorn seeds), a third extract derived from Citrullus lanatus seed (extracts from watermelon seeds), and a fourth extract derived from Phyllanthus emblica fruit (extracts from Indian gooseberry fruit). These ingredients are blended into a single formulation that acts to reduce blood pressure and cholesterol levels and improve blood sugar control, thereby addressing multiple facets of cardiovascular health. By combining these particular ingredients, the composition takes advantage of their complementary and potentially synergistic effects: for example, Rauvolfia serpentina provides potent antihypertensive alkaloids, Phyllanthus emblica contributes cholesterol-lowering and anti-diabetic compounds, Hippophae rhamnoides supplies cardioprotective omega fatty acids and antioxidants, and Citrullus lanatus seeds offer vasodilatory and metabolic benefits. Together, the composition offers a holistic treatment for patients suffering from one or more of these conditions, with the benefit of being derived entirely from natural sources.

In certain embodiments of the invention, the natural ingredients are present in the composition in specific proportional ranges by weight. For example, the first extract derived from Rauvolfia serpentina is present in an amount ranging from 20% to 40% by weight of the composition, the second extract derived from Hippophae rhamnoides seed is present in an amount ranging from 20% to 40% by weight of the composition, the third extract derived from Citrullus lanatus seed is present in an amount ranging from 10% to 30% by weight of the composition, and the fourth extract derived from Phyllanthus emblica fruit is present in an amount ranging from 10% to 30% by weight of the composition. These ranges can be adjusted within the given limits to optimize efficacy; one preferred formulation uses about 30% by weight of the first extract derived from Rauvolfia serpentina, about 30% by weight of the second extract derived from Hippophae rhamnoides seed, about 20% by weight of the third extract derived from Citrullus lanatus seed, and about 20% by weight of the fourth extract derived from Phyllanthus emblica fruit. The composition may optionally include standard pharmaceutical additives (such as excipients or carriers) for formulation stability or delivery, though the active efficacy is derived from the natural ingredient blend.

In another aspect, the present invention encompasses a method for preparing the aforementioned composition. The natural ingredients are processed through a multi-step method to maximize the retention of their active constituents and to ensure the final product's efficacy and stability. In an exemplary embodiment, the method involves: harvesting each of the plant ingredients at optimal maturity; cleaning the raw materials; drying them to a suitable moisture level; grinding the dried materials into fine powders; extracting the powders with a suitable solvent to obtain concentrated plant extracts comprising a first extract derived from Rauvolfia serpentina, a second extract derived from Hippophae rhamnoides seed, a third extract derived from Citrullus lanatus seed, and a fourth extract derived from Phyllanthus emblica fruit; concentrating and drying these extracts to yield dry extract powders; optionally fractionating the extracts to isolate purified bioactive fractions; blending one of the dried extracts and the purified active fractions of the first extract, the second extract, the third extract, and the fourth extract in a weight ratio ranging from 20-40% Rauvolfia serpentina, 20-40% Hippophae rhamnoides seeds, 10-30% Citrullus lanatus seeds, and 10-30% Phyllanthus emblica fruits to obtain a homogeneous mixture of a blended extract; and finally encapsulating or formulating the combined extracts/fractions into a convenient dosage form (such as capsules). Throughout this method, the ingredients can be combined or blended as needed to achieve a homogeneous mixture. This careful preparation method preserves the major effective chemical components of each ingredient and results in a stable, potent composition that can be easily administered to patients.

In yet another aspect, the present invention provides a method of treatment for hypertension, hypercholesterolemia, diabetes, and/or related cardiovascular risk factors. The method comprises administering to a subject in need an effective amount of the described natural ingredient composition. The composition can be formulated into oral dosage forms (for example, capsules or tablets) for ease of administration. Because the ingredients are natural and traditionally used, the therapeutic method offers the advantage of fewer side effects and reduced risk of long-term complications. Patients receiving the composition may experience lowered blood pressure, improved lipid profiles (e.g., reduced LDL cholesterol and triglycerides, increased HDL cholesterol), better blood sugar regulation, and overall reduction in cardiovascular risk markers, all with minimal adverse effects.

Therefore, the present invention provides a novel multi-herbal composition and its preparation, which together offer a natural therapeutic solution to manage and treat hypertension, high cholesterol, diabetes, and associated cardiovascular risks. The following sections and accompanying drawings describe the invention in further detail, including various embodiments and examples, to facilitate a clear understanding of the inventive concepts.

To further clarify the advantages and features of the present invention, a more particular description of the invention will follow by reference to specific embodiments thereof, which are illustrated in the appended figures. It is to be appreciated that these figures depict only typical embodiments of the invention and are therefore not to be considered limiting in scope. The invention will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a block diagram illustrating an embodiment of a composition for a treatment of one or more health conditions, in accordance with an embodiment of the present disclosure; and

FIG. 2 is a flow chart illustrating an embodiment of a method for preparing the composition of FIG. 1, in accordance with an embodiment of the present disclosure.

Further, those skilled in the art will appreciate that elements in the figures are illustrated for simplicity and may not have necessarily been drawn to scale. Furthermore, in terms of the method steps, chemical compounds, and parameters used herein may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more components, compounds, and ingredients preceded by “comprises . . . a” does not, without more constraints, preclude the existence of other components or compounds or ingredients, or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

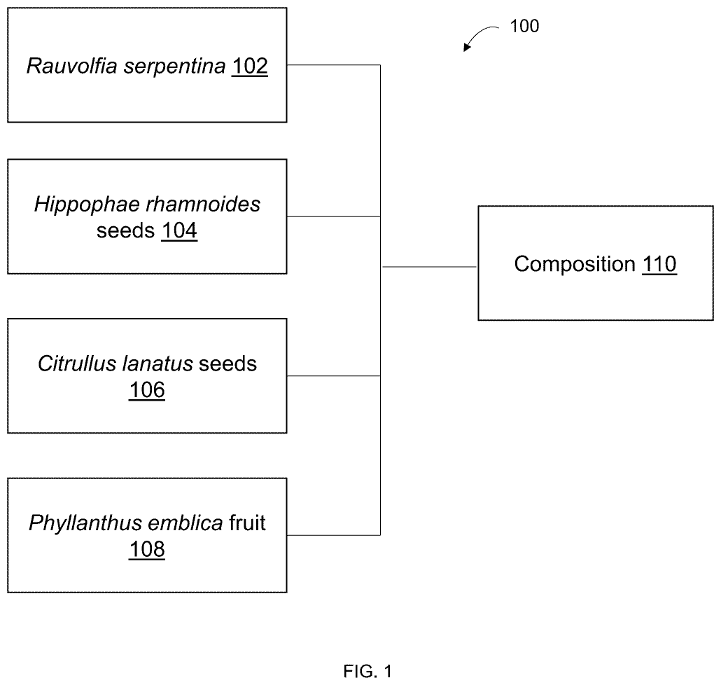

FIG. 1 is a block diagram illustrating an embodiment of a composition for a treatment of one or more health conditions, in accordance with an embodiment of the present disclosure. FIG. 1 illustrates an embodiment of the composition 110 comprising natural ingredients (Rauvolfia serpentina 102, Hippophae rhamnoides seeds 104, Citrullus lanatus seeds 106, and Phyllanthus emblica fruit 108) for treatment of one or more conditions comprising hypertension, high cholesterol, diabetes, and cardiovascular risk factors. In the diagram, each natural ingredient 102, 104, 106, 108 is shown contributing to the composition 110, in accordance with an embodiment of the present disclosure.

The present invention provides a composition and method for preparing the composition and using the composition for treatment of one or more health conditions selected from hypertension, hyperlipidemia, diabetes mellitus, and associated cardiovascular risk factors. The present invention further discloses standardizing and formulating of the composition into pharmaceutically acceptable oral dosage forms. The one or more health conditions comprise hypertension, hyperlipidemia, diabetes mellitus, and cardiovascular risk factors.

Embodiments of the present invention relate to a composition for treatment of one or more health conditions. The composition comprising a first extract derived from Rauvolfia serpentina, a second extract derived from Hippophae rhamnoides seed, a third extract derived from Citrullus lanatus seed, and a fourth extract derived from Phyllanthus emblica fruit.

In another embodiment of the present invention, the first extract derived from Rauvolfia serpentina is present in an amount ranging from 20% to 40% by weight of the composition, the second extract derived from Hippophae rhamnoides seed is present in an amount ranging from 20% to 40% by weight of the composition, the third extract derived from Citrullus lanatus seed is present in an amount ranging from 10% to 30% by weight of the composition, and the fourth extract derived from Phyllanthus emblica fruit is present in an amount ranging from 10% to 30% by weight of the composition.

In one exemplary embodiment of the present invention, the composition comprises about 30% by weight of the first extract derived from Rauvolfia serpentina, about 30% by weight of the second extract derived from Hippophae rhamnoides seed, about 20% by weight of the third extract derived from Citrullus lanatus seed, and 20% by weight of the fourth extract derived from Phyllanthus emblica fruit. The first extract, the second extract, the third extract, and the fourth extract are one of a dried extract and a purified bioactive fraction prepared by solvent extraction of a respective plant material. The composition is formulated as an oral dosage form selected from one of a capsule, a tablet, and a powder-filled sachet. The composition further comprises at least one pharmaceutically acceptable excipients selected from flow agents, fillers, binders, and stabilizers.

In one exemplary embodiment of the present invention, the first extract derived from Rauvolfia serpentina comprises bioactive alkaloids comprising reserpine in safe therapeutic concentrations. The first extract, the second extract, the third extract, and the fourth extract are obtained by solvent extraction using one of ethanol, aqueous ethanol, and supercritical carbon dioxide.

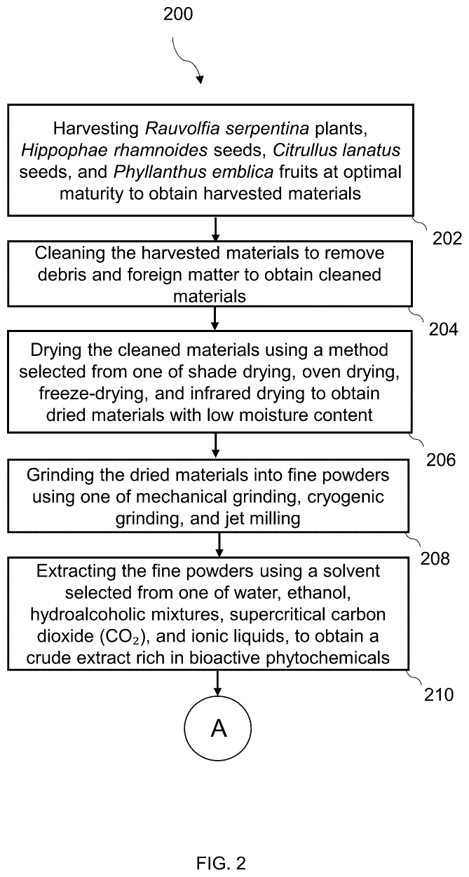

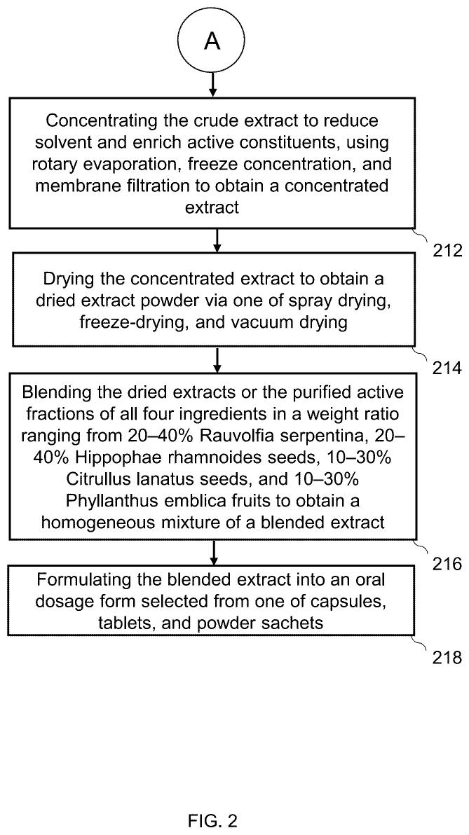

FIG. 2 is a flow chart illustrating an embodiment of a method for preparing the composition, in accordance with an embodiment of the present disclosure. FIG. 2 illustrates an embodiment of a method 200 for preparing the composition of FIG. 1.

In another embodiment of the present invention, a method 200 for preparing a composition for a treatment of one or more health conditions is provided. The method comprises harvesting Rauvolfia serpentina plants, Hippophae rhamnoides seeds, Citrullus lanatus seeds, and Phyllanthus emblica fruits at optimal maturity to obtain harvested materials, at step 202. The harvested materials are cleaned to remove debris and foreign matter to obtain cleaned materials, at step 204. The cleaned materials are dried using a process selected from one of shade drying, oven drying, freeze-drying, and infrared drying to obtain dried materials with a low moisture content, at step 206. The dried materials are ground into fine powders using one of mechanical grinding, cryogenic grinding, and jet milling, at step 208. The fine powders are extracted using a solvent selected from one of water, ethanol, hydroalcoholic mixtures, supercritical carbon dioxide (CO₂), and ionic liquids, to obtain a crude extract rich in bioactive phytochemicals, at step 210. The crude extract is concentrated to reduce solvent and enrich active constituents, using rotary evaporation, freeze concentration, and membrane filtration to obtain a concentrated extract comprising a first extract derived from Rauvolfia serpentina, a second extract derived from Hippophae rhamnoides seed, a third extract derived from Citrullus lanatus seed, and a fourth extract derived from Phyllanthus emblica fruit, at step 212. The concentrated extract is dried to obtain a dried extract powder of the first extract, the second extract, the third extract, and the fourth extract via one of spray drying, freeze-drying, and vacuum drying, at step 214. One of the dried extracts and the purified active fractions of the first extract, the second extract, the third extract, and the fourth extract are blended in a weight ratio ranging from 20-40% Rauvolfia serpentina, 20-40% Hippophae rhamnoides seeds, 10-30% Citrullus lanatus seeds, and 10-30% Phyllanthus emblica fruits to obtain a homogeneous mixture of a blended extract, at step 216. The blended extract is formulated into an oral dosage form selected from one of capsules, tablets, and powder sachets, at step 218.

In another embodiment of the present invention, the method further comprising the step of optionally fractionating the concentrated extract to isolate purified active fractions via one of column chromatography, preparative high-performance liquid chromatography (HPLC), and crystallization. The extraction is carried out by one of maceration, Soxhlet extraction, ultrasonic-assisted extraction, and supercritical fluid extraction. The solvent used for the extraction is selected from one of ethanol, aqueous ethanol, and supercritical carbon dioxide. The solvent is removed by evaporation under one of reduced pressure and vacuum drying. The drying of the extract is carried out by spray drying with a carrier selected from one of maltodextrin and gum arabic. The grinding is performed using at least one of techniques selected from mechanical grinding using one of rotary blade mills and hammer mills, cryogenic grinding to preserve volatile and heat-labile bioactive compounds, ball milling, jet milling, fluid energy milling, and pin milling, to produce a fine powder with a defined particle size for optimal extraction of phytochemicals. The first extract, the second extract, the third extract, and the fourth extract are optionally fractionated by one of chromatographic separation, ultrafiltration, and crystallization. The optional fractionation step isolates one of alkaloid-rich, flavonoid-rich, and polyphenol-rich fractions from at least one of the first extract, the second extract, the third extract, and the fourth extract. The formulation step comprises encapsulating the blended extract into one of gelatin capsules and hydroxypropyl methylcellulose (HPMC) capsules.

In one exemplary embodiment of the present invention, the composition of natural ingredients: Referring to FIG. 1, an embodiment of the invention comprises a composition 110 formed from a combination of natural plant ingredients. Specifically, the composition includes Rauvolfia serpentina 102 (commonly known as Sarpagandha), Hippophae rhamnoides seeds 104 (sea buckthorn seeds), Citrullus lanatus seeds 106 (watermelon seeds), and Phyllanthus emblica 108 (Indian gooseberry, fruit of the amla tree). Each of these components is chosen for its medicinal properties and contributes to the treatment of hypertension, high cholesterol, diabetes, and cardiovascular risk factors. In preferred embodiments, the active portions of these ingredients are used in the form of extracts, concentrates, or purified fractions that contain the key bioactive compounds of each plant. For example, Rauvolfia serpentina contains alkaloids (such as reserpine, ajmaline, serpentine, etc.) known for their antihypertensive and sedative effects; Hippophae rhamnoides seeds contain flavonoids and essential fatty acids (like palmitoleic acid, omega-3 and omega-6 oils) that can improve cholesterol profiles and reduce inflammation; Citrullus lanatus seeds are rich in amino acids like L-arginine and citrulline, as well as minerals such as magnesium, which support vasodilation (thus helping to lower blood pressure) and glucose metabolism; Phyllanthus emblica (amla) is rich in vitamin C, tannins, and other antioxidants that have been shown to lower LDL cholesterol, improve HDL cholesterol, and exert anti-diabetic effects. By combining these four ingredients, the composition provides a synergistic, multi-modal therapeutic effect: it simultaneously addresses elevated blood pressure, abnormal cholesterol levels, and high blood sugar, thereby tackling the major interrelated risk factors for cardiovascular disease.

### Ingredient Profile and Mechanism of Action:

Rauvolfia serpentina Extract: Rauvolfia serpentina extract is the primary antihypertensive agent in the composition. Its alkaloids, principally reserpine, act by depleting catecholamines (like norepinephrine) from peripheral nerve endings. This leads to a reduction in heart rate, relaxation of blood vessels (vasodilation), and a subsequent lowering of blood pressure. The extract is standardized to contain safe yet effective concentrations of these alkaloids.

Hippophae rhamnoides (Sea Buckthorn) Seed Extract: Hippophae rhamnoides Seed extract provides potent antioxidant and lipid-modulating effects. It is rich in flavonoids, tocopherols, and polyunsaturated fatty acids. These compounds protect the cardiovascular system from oxidative stress, a key driver of atherosclerosis. They also help reduce levels of LDL (“bad”) cholesterol and triglycerides while potentially increasing HDL (“good”) cholesterol, thus directly addressing hyperlipidemia.

Citrullus lanatus (Watermelon) Seed Extract: Citrullus lanatus seed extract improves vascular health and blood flow. Its active compound, L-citrulline, is converted in the body to L-arginine, the substrate for nitric oxide synthase (NOS). Nitric oxide (NO) is a powerful vasodilator that relaxes the endothelium (the inner lining of blood vessels), improving circulation and helping to lower blood pressure. This provides a complementary mechanism to the action of Rauvolfia serpentina.

Phyllanthus emblica (Amla) Fruit Extract: Phyllanthus emblica fruit extract offers broad-spectrum metabolic and protective benefits. It is one of the richest natural sources of Vitamin C and contains a complex of tannins and polyphenols. It enhances endothelial function, reduces oxidative stress, improves insulin sensitivity (addressing diabetes), and has demonstrated lipid-lowering properties. It acts as a powerful systemic antioxidant, protecting organs from damage related to high blood sugar and lipids.

### The Synergistic Effect:

The composition is designed so that the ingredients work in concert. For example, while Rauvolfia lowers blood pressure through neurological pathways, Citrullus lowers it through improved vascular function. While Hippophae directly lowers lipids, Phyllanthus improves the body's overall metabolic handling of both lipids and glucose. This multi-pronged approach reduces the burden on any single physiological pathway and creates a more balanced and potent therapeutic effect.

In one exemplary embodiment of the present invention, the composition of the present invention is intended for oral administration. A typical therapeutic dosage ranges from 500 mg to 2000 mg per day, taken in one or two divided doses. For individuals with mild to moderate hypertension or hyperlipidemia, the composition may be used as a standalone long-term supplement. For patients with more severe conditions, it can be used as an adjunct to conventional therapy, potentially allowing for lower doses of synthetic drugs and reducing their side effect profile.

Importantly, since the composition is derived entirely from natural sources traditionally used in medicine, it is expected to have a favorable safety profile. The natural ingredients help avoid the adverse side effects associated with many synthetic drugs. In particular, the composition is designed to not cause significant side effects, dependency risks, or tolerance development. Patients can potentially use this composition long-term to manage chronic conditions without the complications of synthetic pharmaceuticals. Furthermore, the use of plant-based ingredients is environmentally sustainable and ethically acceptable, as it relies on renewable natural resources.

The present invention is explained further in the following specific examples, which are only by way of illustration and are not to be construed as limiting the scope of the invention.

### Example 1: Capsule Formulation (30/30/20/20 Ratio)

Dried and powdered extracts of the four botanicals, prepared using 70% aqueous ethanol extraction and spray drying, were procured.

The extracts were weighed as follows to prepare a 1 kg batch:
Powder of the first extract derived from Rauvolfia serpentina: 300 g (30%)
Powder of the second extract derived from Hippophae rhamnoides seed: 300 g (30%)
Powder of the third extract derived from Citrullus lanatus seed: 200 g (20%)
Powder of the fourth extract derived from Phyllanthus emblica fruit: 200 g (20%)

The powders were blended in a V-blender for 30 minutes. Colloidal silicon dioxide (1%) and magnesium stearate (0.5%) were added and blended for another 5 minutes.

The final blend was encapsulated into size “00” hydroxypropyl methylcellulose (HPMC) capsules, with each capsule containing 500 mg of the active blend.

### Example 2: Tablet Formulation (40/20/20/20 Ratio)

A batch of the active composition is prepared with a higher concentration of the primary antihypertensive agent. The extracts are weighed as follows:
Powder of the first extract derived from Rauvolfia serpentina: 400 g (40%)
Powder of the second extract derived from Hippophae rhamnoides seed: 200 g (20%)
Powder of the third extract derived from Citrullus lanatus seed: 200 g (20%)
Powder of the fourth extract derived from Phyllanthus emblica fruit: 200 g (20%)

The 1 kg blend of active extracts is mixed with pharmaceutically acceptable excipients suitable for direct compression, including microcrystalline cellulose (as a binder/filler), croscarmellose sodium (as a superdisintegrant), and magnesium stearate (as a lubricant).

The final mixture is compressed into 1000 mg tablets using a rotary tablet press. Each tablet provides a higher unit dose for patients requiring more intensive therapy.

### Example 3: Powder Sachet Formulation with Supercritical CO₂ Extracts

To maximize purity and avoid solvent residues, extracts are obtained using supercritical carbon dioxide (CO₂) extraction.

The dried, purified extracts are weighed to form a composition with enhanced antioxidant focus:
Powder of the first extract derived from Rauvolfia serpentina: 250 g (25%)
Powder of the second extract derived from Hippophae rhamnoides seed: 350 g (35%)
Powder of the third extract derived from Citrullus lanatus seed: 150 g (15%)
Powder of the fourth extract derived from Phyllanthus emblica fruit: 250 g (25%)

The 1 kg blend is mixed with a water-soluble carrier like maltodextrin and a natural flavouring agent.

The final powder is filled into single-dose sachets, each containing 2000 mg of the formulation, intended to be dissolved in 150-200 ml of water before consumption. This dosage form is suitable for patients who have difficulty swallowing pills.

### Example 4: Formulation Using Purified Bioactive Fractions

Instead of using whole extracts, this embodiment utilizes fractions enriched with specific bioactive compounds, as provided by claim 15.
An alkaloid-rich fraction is isolated from Rauvolfia serpentina via column chromatography.
A flavonoid-rich fraction is isolated from Hippophae rhamnoides.
A citrulline-enriched fraction is obtained from Citrullus lanatus.
A polyphenol-rich fraction is prepared from Phyllanthus emblica.

These purified fractions are dried and blended in the 30/30/20/20 weight ratio to create a highly potent 500 mg capsule. This method allows for greater standardization and reduces the overall pill burden for the patient.

Preparation method: Referring to FIG. 2, the present invention further provides a method 200 for producing the aforementioned herbal composition. The method ensures that the active constituents of each ingredient are preserved and that the final product is a homogenous blend of the ingredients in a potent form. In an exemplary embodiment, the method comprises the following steps:

Harvesting 202: Harvest the natural ingredient sources (Rauvolfia serpentina plants, Hippophae rhamnoides seeds, Citrullus lanatus seeds, and Phyllanthus emblica fruits) at optimal maturity. Harvest timing is chosen to maximize the concentration of active phytochemicals in each plant material.

Cleaning 204: Clean the harvested materials to remove any dirt, debris, or foreign matter. This may involve washing with water or appropriate food-grade cleaning solutions and drying the materials after washing, ensuring only the pure plant parts proceed to the next stage.

Drying 206: Dry the cleaned ingredients until a desired low moisture content is achieved. Drying can be performed using one or more of various methods, including but not limited to air drying or shade drying at ambient conditions, sun drying, oven drying at controlled temperatures, freeze-drying (lyophilization), microwave drying, or infrared drying. The drying method is selected to effectively preserve sensitive bioactive compounds (for example, freeze drying can preserve heat-labile constituents). Drying prevents spoilage and facilitates grinding and extraction by reducing moisture.

Grinding 208: Grind the dried plant materials into a fine powder. Grinding can be accomplished using mechanical mills or grinders; suitable techniques include mechanical grinding with blades, ball milling, jet milling (fluid energy milling), cryogenic grinding (grinding at low temperatures to preserve volatile compounds), hammer milling, or pin milling. The grinding process is controlled to achieve a specified particle size range of the powder, which increases the surface area for efficient extraction of phytochemicals.

Extraction 210: Extract the powdered plant materials with a solvent to obtain concentrated extracts containing the active constituents. In some embodiments, the powders of all ingredients can be mixed together and extracted as a blend; in other embodiments, each ingredient is extracted separately and the extracts will be combined later. The solvent for extraction is chosen based on its ability to dissolve the desired compounds and may include, for example, water; organic solvents such as ethanol or methanol; hydroalcoholic mixtures (water-alcohol combinations); supercritical fluids like supercritical CO₂; or ionic liquids. Extraction can be performed by techniques such as maceration, percolation, reflux extraction, Soxhlet extraction, ultrasonic extraction, or supercritical fluid extraction, depending on the solvent and desired efficiency. The result of this step is a crude extract (liquid) containing the soluble bioactive compounds from the plant materials.

Concentration 212: Concentrate the extract to remove excess solvent and increase the potency of the extract to obtain a concentrated extract comprising a first extract derived from Rauvolfia serpentina, a second extract derived from Hippophae rhamnoides seed, a third extract derived from Citrullus lanatus seed, and a fourth extract derived from Phyllanthus emblica fruit. This can be achieved by evaporation or distillation (for instance, using a rotary evaporator under reduced pressure to gently remove solvents at low temperature), freeze concentration (freezing the extract and removing ice to concentrate solutes), or membrane filtration techniques such as ultrafiltration or nanofiltration to remove solvent and small impurities. The concentration step yields a more viscous or smaller-volume extract that contains a higher density of active ingredients.

Drying Extract to Powder 214: Dry the concentrated extract to obtain a solid dried extract powder of the first extract, the second extract, the third extract, and the fourth extract. Various drying methods can be used at this stage, including spray drying (spraying the liquid extract into a hot air chamber to instantaneously dry it into a powder), freeze-drying (to sublimate remaining solvent and preserve structure), vacuum drying, drum drying (spreading the extract on a heated drum), or tray drying in a drying oven. The goal is to produce a stable dry powder of the extract that is suitable for long-term storage and formulation. This powder contains the concentrated active compounds of the original ingredients.

Blending and Homogenization 216: Throughout the above process, and especially after obtaining the dried extracts and the purified active fractions of the first extract, the second extract, the third extract, and the fourth extract, the materials are blended to ensure a uniform composition to obtain a homogeneous mixture of a blended extract. In an embodiment, the individual extracts of each ingredient are combined in the desired proportions and mixed thoroughly so that the actives are evenly distributed. This blending may occur before encapsulation (mixing powders) or even at earlier stages (for instance, combining liquids prior to final drying). The result is a homogeneous composition wherein each dosage unit (e.g. each capsule) contains all four ingredients in consistent ratios.

Encapsulation/Formulation 218: Encapsulate the blended extract to create the final dosage form. The term “encapsulating” here broadly encompasses integrating the active ingredients into a stable delivery vehicle. In one embodiment, the dried extract powders or purified fractions from all ingredients are blended together to form a homogeneous mixture (if not already combined earlier). This blended powder can then be filled into capsules (such as gelatin or vegetarian capsules) in measured doses. In other embodiments, the mixture may be compressed into tablets with suitable binders, or it may be processed into other oral dosage forms. Techniques such as spray-drying encapsulation (spray drying the extract with a carrier like maltodextrin to form microcapsules), extrusion-spheronization (to form pellets), coacervation (to form microcapsules with polymer coatings), or incorporation into liposomes (creating lipid vesicles containing the extract) can also be employed to encapsulate or coat the active ingredients, thereby protecting them and controlling their release. Ultimately, this step produces the final composition in a form that can be conveniently administered to patients (for example, as capsules containing the blended extract of the four ingredients).

Fractionation: Optionally, subject the dried extract or the concentrated extract to a fractionation process to isolate purified fractions enriched in specific active constituents. Fractionating can be accomplished by techniques such as column chromatography (e.g., using silica gel or resin to separate components by polarity or size), preparative HPLC, distillation (if volatile components are targeted), crystallization (to purify specific compounds), or chemical precipitation of certain fractions. This step can yield purified sub-extracts, such as an alkaloid-rich fraction from Rauvolfia or a flavonoid-rich fraction from Phyllanthus. These purified fractions can enhance the consistency and potency of the final composition by ensuring key actives are present in known amounts.

By following these steps, the process yields a stable, potent herbal composition that preserves the major effective chemical components of the original natural ingredients. The careful drying and encapsulation of extracts ensure that the active compounds remain stable over time (for example, encapsulation can protect sensitive constituents from light or oxidation) and that they are delivered efficiently upon ingestion. The process can be adjusted or optimized by those skilled in the art—for instance, selecting a particular solvent for extraction to target certain compounds, or choosing a drying method that best preserves a specific ingredient's actives—without departing from the essence of the invention.

Preferred Composition Ratios: As noted, the composition comprises all four active ingredients in particular proportion ranges. An exemplary formulation by weight is as follows: about 20-40% Rauvolfia serpentina (e.g., root extract), about 20-40% Hippophae rhamnoides (seed extract), about 10-30% Citrullus lanatus (seed extract), and about 10-30% Phyllanthus emblica (fruit extract). These ranges reflect that Rauvolfia and Hippophae may be used in somewhat higher proportions relative to Citrullus and Phyllanthus, likely because the former two provide primary antihypertensive and lipid-lowering effects, respectively, whereas the latter two support and enhance those effects. Within these ranges, the composition can be tailored; for example, one embodiment uses approximately 30% of each Rauvolfia and Hippophae extracts and approximately 20% of each Citrullus and Phyllanthus extracts, yielding a 30:30:20:20 ratio by weight. In another embodiment, a slightly higher proportion of Rauvolfia (e.g., 40%) may be used when hypertension control is the primary goal, or a higher proportion of Phyllanthus emblica may be used for patients where blood sugar control is equally important. The flexibility of these ratios allows customization of the composition to individual needs while remaining within the scope of the invention.

It should be noted that the composition may consist essentially of the four specified plant-derived extracts, or it may further include innocuous carriers and excipients to facilitate manufacturing and administration. For example, a small amount of a flow agent, filler, or stabilizer (such as microcrystalline cellulose, magnesium stearate, or silica) can be added when filling capsules or pressing tablets, without affecting the therapeutic activity. The term “composition” as used herein encompasses both the pure blend of active extracts and such formulations with pharmaceutically acceptable additives.

Dosage and Administration: The herbal composition of the present invention can be administered to patients in need of therapy for hypertension, high cholesterol, diabetes, or prevention of cardiovascular events. The typical route of administration is oral. For instance, the composition can be delivered in capsule form. Each capsule might contain, for example, about 500 milligrams of the combined extract (with the ingredients in the ratios described above). A recommended dosage regimen could be one to two capsules daily (approximately 500 mg to 1000 mg per day of active ingredients), taken with water. The dosage can be adjusted by healthcare providers based on the severity of the condition and the patient's response; some patients may benefit from higher doses (e.g., up to 1500 mg or 2000 mg per day, split into multiple doses), while others may maintain control of their blood pressure, cholesterol, and glucose with a lower dose (e.g., 250 mg per day). Because the composition is natural and well-tolerated, it can be used as a long-term daily supplement or treatment. It may be used alone as a monotherapy or adjunctively alongside other medications (with medical supervision) to potentially reduce the required doses of synthetic drugs. The composition's multi-faceted action (antihypertensive, hypolipidemic, and hypoglycemic) makes it especially useful for patients with metabolic syndrome or other combinations of cardiovascular risk factors.

The inventive composition offers several key therapeutic advantages. First, it addresses multiple conditions simultaneously: a single preparation can help lower blood pressure, improve lipid profiles, and regulate blood sugar. This broad-spectrum efficacy can simplify patient treatment plans, improving compliance (patients prefer taking fewer medications). Second, the risk of side effects is reduced. Each ingredient is derived from edible or medicinal plants with long histories of human use, suggesting a wide safety margin. For example, Phyllanthus emblica (amla) is commonly consumed as a dietary fruit and is known for its health benefits with no significant adverse effects; Hippophae rhamnoides is used in nutritional supplements and traditional remedies; Citrullus lanatus seeds are eaten as snacks and are rich in nutrients; Rauvolfia serpentina is potent but used in controlled extract form in the composition at safe concentrations. By using the whole-plant extracts or fractions (rather than a single isolated chemical), supportive phytochemicals in each ingredient may mitigate potential side effects (for instance, the presence of various alkaloids and compounds in Rauvolfia extract might allow effective blood pressure reduction with a lower dose of reserpine than a pure drug, reducing risk of depression or sedation that pure reserpine can cause). Third, the natural composition may provide ancillary health benefits beyond the targeted conditions, such as antioxidant effects that reduce oxidative stress or anti-inflammatory effects that improve vascular function. This holistic approach can improve overall cardiovascular health, not just the numeric measures of blood pressure or cholesterol.

From a manufacturing and environmental perspective, the use of renewable plant resources and green extraction methods (e.g., using water or ethanol which are relatively environmentally friendly solvents, or supercritical CO₂ which leaves no solvent residue) makes the production of this composition sustainable and with minimal chemical waste compared to synthetic drug manufacturing. The resulting product is an ethically sourced, eco-friendly therapeutic option.

The detailed description above provides exemplary embodiments of the composition and its preparation. These examples are intended to illustrate the principles of the invention and the best mode of practicing it, but they do not limit the scope of the invention. Variations and modifications will be apparent to those skilled in the art in light of this disclosure. For instance, equivalent extraction techniques or alternative formulation approaches can be employed without departing from the core idea of combining these specific natural ingredients for the stated therapeutic purpose. All such variants are intended to be within the scope of the invention as defined in the claims.

The present invention thus provides a novel, standardized, and synergistic herbal composition that combines the therapeutic properties of Rauvolfia serpentina, Hippophae rhamnoides seed, Citrullus lanatus seed, and Phyllanthus emblica fruit extracts in specific proportions. The composition is designed for the effective treatment of chronic conditions such as hypertension, hyperlipidemia, diabetes mellitus, and associated cardiovascular risk factors. The invention also discloses a comprehensive method for the preparation of the composition, involving optimized extraction, concentration, drying, and formulation processes that preserve the integrity and efficacy of bioactive phytoconstituents.

The formulation offers a multi-targeted therapeutic approach with the added benefits of natural origin, reduced side effects, improved patient compliance, and suitability for long-term use either as a standalone supplement or as an adjunct to conventional pharmacotherapy. The disclosed process and composition are scalable and adaptable for commercial manufacturing under good manufacturing practices (GMP) and can be standardized to ensure reproducibility and efficacy.

Definition of Terms: In interpreting this disclosure and the claims that follow, the terms “comprise” or “comprising” are used in an open-ended sense, such that inclusion of stated elements, steps, or ingredients does not preclude the presence of additional ones. Similarly, the terms “including”, “containing”, or “having” are to be interpreted equivalently to “comprising”. The singular forms “a”, “an”, and “the” include plural references unless the context clearly dictates otherwise (thus, reference to “a plant extract” encompasses one or more such extracts). The term “effective amount” refers to an amount of a composition sufficient to produce a desired therapeutic effect (e.g., lowering blood pressure or cholesterol) in a subject. All percentages given are by weight, unless otherwise specified. It is also to be understood that when an element or step is described as “preferred” or “particularly preferred”, it is optional and does not limit the scope of the invention or claims—rather, it indicates one example of a beneficial embodiment.

The detailed description and example embodiments are intended to be illustrative rather than restrictive. It will be apparent to those skilled in the art that various modifications and variations can be made to the composition, its method of preparation, and its application without departing from the spirit or scope of the invention. For instance, variations in excipients, minor adjustments to extraction parameters, or the inclusion of other synergistic herbal agents may be made. Therefore, the invention is not to be limited to the embodiments disclosed herein but is to be accorded the widest scope consistent with the principles and features disclosed and claimed.

While specific language has been used to describe the invention, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

What is claimed is:

1. A composition for a treatment of one or more health conditions, the composition comprising:
a first extract derived from Rauvolfia serpentina;
a second extract derived from Hippophae rhamnoides seed;
a third extract derived from Citrullus lanatus seed; and
a fourth extract derived from Phyllanthus emblica fruit.

2. The composition of claim 1, wherein the first extract derived from Rauvolfia serpentina is present in an amount ranging from 20% to 40% by weight of the composition, the second extract derived from Hippophae rhamnoides seed is present in an amount ranging from 20% to 40% by weight of the composition, the third extract derived from Citrullus lanatus seed is present in an amount ranging from 10% to 30% by weight of the composition, and the fourth extract derived from Phyllanthus emblica fruit is present in an amount ranging from 10% to 30% by weight of the composition.

3. The composition of claim 2, wherein the composition comprises about 30% by weight of the first extract derived from Rauvolfia serpentina, about 30% by weight of the second extract derived from Hippophae rhamnoides seed, about 20% by weight of the third extract derived from Citrullus lanatus seed, and 20% by weight of the fourth extract derived from Phyllanthus emblica fruit.

4. The composition of claim 1, wherein the first extract, the second extract, the third extract, and the fourth extract are one of a dried extract and a purified bioactive fraction prepared by solvent extraction of a respective plant material.

5. The composition of claim 1, wherein one or more health conditions comprise hypertension, hyperlipidemia, diabetes mellitus, and cardiovascular risk factors.

6. The composition of claim 1, wherein the composition is formulated as an oral dosage form selected from one of a capsule, a tablet, and a powder-filled sachet.

7. The composition of claim 1, further comprising at least one pharmaceutically acceptable excipients selected from flow agents, fillers, binders, and stabilizers.

8. The composition of claim 1, wherein the first extract derived from Rauvolfia serpentina comprises bioactive alkaloids comprising reserpine in safe therapeutic concentrations.

9. The composition of claim 1, wherein the first extract, the second extract, the third extract, and the fourth extract are obtained by solvent extraction using one of ethanol, aqueous ethanol, and supercritical carbon dioxide.

10. A method for preparing a composition for a treatment of one or more health conditions, the method comprising:
harvesting Rauvolfia serpentina plants, Hippophae rhamnoides seeds, Citrullus lanatus seeds, and Phyllanthus emblica fruits at optimal maturity to obtain harvested materials;
cleaning the harvested materials to remove debris and foreign matter to obtain cleaned materials;
drying the cleaned materials using a process selected from one of shade drying, oven drying, freeze-drying, and infrared drying to obtain dried materials with a low moisture content;
grinding the dried materials into fine powders using one of mechanical grinding, cryogenic grinding, and jet milling;
extracting the fine powders using a solvent selected from one of water, ethanol, hydroalcoholic mixtures, supercritical carbon dioxide (CO₂), and ionic liquids, to obtain a crude extract rich in bioactive phytochemicals;
concentrating the crude extract to reduce solvent and enrich active constituents, using rotary evaporation, freeze concentration, and membrane filtration to obtain a concentrated extract comprising a first extract derived from Rauvolfia serpentina, a second extract derived from Hippophae rhamnoides seed, a third extract derived from Citrullus lanatus seed, and a fourth extract derived from Phyllanthus emblica fruit;
drying the concentrated extract to obtain a dried extract powder of the first extract, the second extract, the third extract, and the fourth extract via one of spray drying, freeze-drying, and vacuum drying;
blending one of the dried extracts and the purified active fractions of the first extract, the second extract, the third extract, and the fourth extract in a weight ratio ranging from 20-40% Rauvolfia serpentina, 20-40% Hippophae rhamnoides seeds, 10-30% Citrullus lanatus seeds, and 10-30% Phyllanthus emblica fruits to obtain a homogeneous mixture of a blended extract; and
formulating the blended extract into an oral dosage form selected from one of capsules, tablets, and powder sachets.

11. The method of claim 10, wherein the drying of the extract is carried out by spray drying with a carrier selected from one of maltodextrin and gum arabic.

12. The method of claim 10, wherein the grinding is performed using at least one of techniques selected from mechanical grinding using one of rotary blade mills and hammer mills, cryogenic grinding to preserve volatile and heat-labile bioactive compounds, ball milling, jet milling, fluid energy milling, and pin milling, to produce a fine powder with a defined particle size for optimal extraction of phytochemicals.

13. The method of claim 10, wherein the extraction step is carried out by one of maceration, Soxhlet extraction, ultrasonic-assisted extraction, and supercritical fluid extraction.

14. The method of claim 10, wherein the solvent used for the extraction step is selected from one of ethanol, aqueous ethanol, and supercritical carbon dioxide.

15. The method of claim 10, wherein the solvent is removed by evaporation under one of reduced pressure and vacuum drying.

16. The method of claim 10, further comprising a step of fractionating the concentrated extract to isolate purified active fractions via one of column chromatographic separation, ultrafiltration, preparative high-performance liquid chromatography (HPLC), and crystallization.

17. The method of claim 16, wherein the optional fractionation step isolates one of alkaloid-rich, flavonoid-rich, and polyphenol-rich fractions from at least one of the first extract, the second extract, the third extract, and the fourth extract.

18. The method of claim 10, wherein the formulation step comprises encapsulating the blended extract into one of gelatin capsules and hydroxypropyl methylcellulose (HPMC) capsules.

19. A method for treatment of one or more health conditions, the method comprising administering to a patient a therapeutically effective amount of the composition of claim 1.

20. The method for treatment of claim 19, wherein the therapeutically effective amount of the composition administered orally as a dosage form is about 500 mg to about 2000 mg per day, optionally in divided doses.

21. The method for treatment of claim 19, wherein the composition is administered as one of a long-term daily supplement and as an adjunct to conventional therapy.
